(19) United States
(12) Patent Application Publication
(10) Pub. No.: US 2023/0033419 A1
(43) Pub. Date: Feb. 2, 2023

(54) In-Line Winterization Process for Plant Extracts

(71) Applicant: Green Mill Supercritical, Inc., Pittsburgh, PA (US)

(72) Inventors: Jesse R. Turner, Erie, CO (US); Jeremy M. Diehl, Pittsburgh, PA (US)

(21) Appl. No.: 17/870,066

(22) Filed: Jul. 21, 2022

Related U.S. Application Data
(60) Provisional application No. 63/224,191, filed on Jul. 21, 2021.

(51) Int. Cl. A61K 36/185; A61K 31/05; A61K 31/352; A61K 31/60

(52) U.S. Cl. CPC A61K 36/185; A61K 31/05; A61K 31/352; A61K 31/60; A61K 2236/37; A61K 2236/53

(57) ABSTRACT

A process for extracting a target compound from a biomass includes: extracting a compound from a biomass charge using supercritical or subcritical CO₂ as a solvent, at a first pressure and a first temperature, to yield a compound-laden solvent stream, the compound-laden solvent stream including the solvent, a solubilized target compound, and a solubilized byproduct compound; changing the temperature while maintaining the first pressure of the compound-laden solvent stream to desolubilize the byproduct compound from the compound-laden solvent stream to yield a resultant stream containing the solvent, the solubilized target compound, and a desolubilized byproduct compound; passing the resultant stream through a filter medium to separate the desolubilized byproduct compound from the solubilized target compound; and collecting the solubilized target compound.

### BACKGROUND

### Field

The present disclosure is directed to a process for producing a refined oil product from a biomass by removing compounds such as waxes and lipids.

### Technical Considerations

Recently, there has been increased interest by consumers in extracting various organic compounds from plants for human use and consumption. For example, while extracting cannabidiol (CBD), tetrahydrocannabinol (THC), other cannabinoids, terpenes, terpenoid compounds, or other botanical compounds by way of supercritical carbon dioxide (CO₂) from cannabis, hops, and other botanical biomass, byproduct compounds such as various waxes and lipids are frequently extracted as well. These byproduct compounds are not desired because of their effects on the purity and properties of the final extract.

To avoid the detrimental effect of the waxes and wax-like compounds including n-alkanes, paraffins, alkyl esters, and fatty acid compounds, it is known to perform winterization after extraction is complete. During conventional winterization, the extract is contacted by an alcohol solvent such as ethanol at temperatures of about −60° C. to about −20° C., which desolubilizes and removes the plant waxes from the extract. However, this process is costly and time-consuming because it must be conducted after the extraction is otherwise complete. This process can take up to several days to complete Furthermore, the alcohol solvents such as ethanol that are used must be recovered to ensure the quality of the final product, as well as for economic and environmental reasons.

In-line winterization attempts to avoid the above steps of contacting the extract with alcohol solvent, instead adjusting the process conditions and configuration of existing supercritical CO₂ extraction processes. However, current iterations of in-line winterization suffer from cannabinoid loss when the temperature and pressure is reduced in a collection vessel intended to precipitate the waxes. This occurs because the target (desired) extract compounds (including CBD, THC, and other cannabinoids and their acid forms) desolubilize and collect with the waxes. There is a need for an improved in-line winterization process that avoids the problems of conventional techniques, thereby enabling extraction of target compounds from plant matter without contamination by plant waxes in an efficient, cost-effective manner. The present disclosure is aimed at solving this and other problems related to traditional winterization techniques.

### SUMMARY OF THE DISCLOSURE

According to some non-limiting aspects of the disclosure, a process for extracting a target compound from a biomass includes: extracting a compound from a biomass charge using supercritical or subcritical CO₂ as a solvent, at a first pressure and a first temperature, to yield a compound-laden solvent stream, the compound-laden solvent stream including the solvent, a solubilized target compound, and a solubilized byproduct compound; changing the temperature while maintaining the first pressure of the compound-laden solvent stream to desolubilize the byproduct compound from the compound-laden solvent stream to yield a resultant stream containing the solvent, the solubilized target compound, and a desolubilized byproduct compound; passing the resultant stream through a filter medium to separate the desolubilized byproduct compound from the solubilized target compound; and collecting the solubilized target compound.

In some non-limiting aspects, the process may include diluting the compound-laden solvent stream with a second solvent stream while maintaining the first pressure. The process may include decreasing the temperature of the diluted compound-laden solvent stream while maintaining the first pressure to desolubilize the byproduct compound from the diluted compound-laden solvent stream. The second solvent stream may include supercritical or subcritical CO₂.

In some non-limiting aspects, the biomass charge may be selected from a plant or fungal species. The biomass charge may include cannabis. The target compound may be extracted from at least one of plants or trees including Cassia, Cinnamon, Sassafras, Camphor, Cedar, Rosewood, Sandalwood, Agarwood, Galangal, Ginger, Basil, Bay Leaf, Buchu, Cannabis, Cinnamon, Sage, Eucalyptus, Guava, Lemon grass, Midaleuca, Oregano, Patchouli, Peppermint, Pine, Rosemary, Spearmint, Tea tree, Thyme, Tsuga, Wintergreen, Benzoin, Copaiba, Frankincense, Myrrh, Chamomile, Clary sage, Clove, Scented geranium, Hops, Hyssop, Jasmine, Lavender, Manuka, Marjoram, Orange, Rose, Ylang-ylang, Bergamot, Grapefruit, Lemon, Lime, Orange, Mango, Tangerine, Valerian, Berries including Allspice and Juniper; Seeds including Anise, Buchu, Celery, Cumin, Nutmeg oil; or truffles. The target compound may be selected from at least one of cannabinoids, tetrahydrocannabinol (THC), cannabinoid isomers, cannabinoid stereoisomers, tetrahydrocannabinolic acid (THCA), cannabidiol (CBD), cannabidiolic acid (CBDA), cannabinol, (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannebielsoin (CBE), cannabicitran (CBT), or combinations or derivatives thereof. The byproduct compound may include a wax or a lipid.

In some non-limiting aspects, the first temperature may be from −60° C. to 200° C. The first pressure may be from 400 psi to 25,000 psi. The first temperature may be from 0° C. to 100° C., and the first pressure may be from 800 psi to 7,500 psi. The changing the temperature may include decreasing the temperature to from −10° C. to 15° C. The changing the temperature may include decreasing the temperature to from 0° C. to 6° C. The process may include reducing the pressure of the solubilized target compound to release the target compound from the solvent. The released target compound may be free of the byproduct compound.

According to some non-limiting aspects of the disclosure, a target compound is formed which is extracted from a biomass charge according to a process described herein, where the target compound is free of a wax or a lipid.

According to some non-limiting aspects of the disclosure, a process for producing a refined oil product from a biomass includes: extracting a compound from a biomass charge using a first solvent stream including a solvent, the first solvent stream having a first pressure of from 400 psi to 25,000 psi and a first temperature to produce a first oil extract dissolved in the solvent; mixing the first oil extract dissolved in the solvent with a second solvent stream while maintaining the first pressure to form a diluted oil extract; and delivering the diluted oil extract to a filter to separate a byproduct compound from the diluted oil extract to form a dilute refined oil including a target compound solubilized in the solvent.

In some non-limiting aspects, mixing the first oil extract dissolved in the solvent with the second solvent stream includes maintaining the first pressure while reducing the temperature of the first oil extract dissolved in the solvent below the first temperature by the second solvent stream. The process may include delivering the dilute oil extract to a heat exchanger to reduce the temperature of the dilute oil extract below the first temperature while maintaining the first pressure to desolubilize the byproduct compound.

The following numbered clauses are illustrative of various aspects of the disclosure:

Clause 1: A process for extracting a target compound from a biomass, the process comprising: extracting a compound from a biomass charge using supercritical or subcritical CO₂ as a solvent, at a first pressure and a first temperature, to yield a compound-laden solvent stream, the compound-laden solvent stream comprising the solvent, a solubilized target compound, and a solubilized byproduct compound; changing the temperature while maintaining the first pressure of the compound-laden solvent stream to desolubilize the byproduct compound from the compound-laden solvent stream to yield a resultant stream containing the solvent, the solubilized target compound, and a desolubilized byproduct compound; passing the resultant stream through a filter medium to separate the desolubilized byproduct compound from the solubilized target compound; and collecting the solubilized target compound.

Clause 2: The process of clause 1, further comprising diluting the compound-laden solvent stream with a second solvent stream while maintaining the first pressure, wherein optionally the second solvent stream has a temperature lower than the first temperature.

Clause 3: The process of clause 2, further comprising decreasing the temperature of the diluted compound-laden solvent stream while maintaining the first pressure to desolubilize the byproduct compound from the diluted compound-laden solvent stream.

Clause 4: The process of clause 2 or 3, wherein the second solvent stream comprises supercritical or subcritical CO₂.

Clause 5: The process of any of clauses 1-4, wherein the biomass charge is selected from a plant or fungal species.

Clause 6: The process of any of clauses 1-5, wherein the biomass charge comprises cannabis.

Clause 7: The process of any of clauses 1-6, wherein the target compound is extracted from at least one of plants or trees including Cassia, Cinnamon, Sassafras, Camphor, Cedar, Rosewood, Sandalwood, Agarwood, Galangal, Ginger, Basil, Bay Leaf, Buchu, Cannabis, Cinnamon, Sage, Eucalyptus, Guava, Lemon grass, Midaleuca, Oregano, Patchouli, Peppermint, Pine, Rosemary, Spearmint, Tea tree, Thyme, Tsuga, Wintergreen, Benzoin, Copaiba, Frankincense, Myrrh, Chamomile, Clary sage, Clove, Scented geranium, Hops, Hyssop, Jasmine, Lavender, Manuka, Marjoram, Orange, Rose, Ylang-ylang, Bergamot, Grapefruit, Lemon, Lime, Orange, Mango, Tangerine, Valerian, Berries including Allspice and Juniper; Seeds including Anise, Buchu, Celery, Cumin, Nutmeg oil; or truffles.

Clause 8: The process of any of clauses 1-7, wherein the target compound is selected from at least one of cannabinoids, tetrahydrocannabinol (THC), cannabinoid isomers, cannabinoid stereoisomers, tetrahydrocannabinolic acid (THCA), cannabidiol (CBD), cannabidiolic acid (CBDA), cannabinol, (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannebielsoin (CBE), cannabicitran (CBT), or combinations or derivatives thereof.

Clause 9: The process of any of clauses 1-8, wherein the byproduct compound comprises a wax or a lipid.

Clause 10: The process of any of clauses 1-9, wherein the first temperature is from −60° C. to 200° C., such as from −60° C.-100° C., from 0° C. to 100° C., from 32° C. to 100° C., or from 20° C. to 32° C.

Clause 11: The process of any of clauses 1-10, wherein the first pressure is from 400 psi to 25,000 psi, such as from 400 to 20,000 psi, from 400 to 10,000 psi, from 800 to 5,000 psi, or from 1,000 to 10,000 psi.

Clause 12: The process of any of clauses 1-11, wherein the first temperature is from 0° C. to 100° C. and, the first pressure is from 800 psi to 7,500 psi, such as from 800 psi to 6,000 psi or from 800 psi to 5,000 psi.

Clause 13: The process of any of clauses 1-12, wherein the changing the temperature comprises decreasing the temperature to from −10° C. to 15° C.

Clause 14: The process of any of clauses 1-13, wherein the changing the temperature comprises decreasing the temperature to from 0° C. to 6° C.

Clause 15: The process of any of clauses 1-14, further comprising reducing the pressure of the solubilized target compound to release the target compound from the solvent.

Clause 16: The process of clause 15, wherein the released target compound is free of the byproduct compound.

Clause 17: A target compound extracted from a biomass charge according to the process of any of clauses 1-16, wherein the target compound is free of a wax or a lipid.

Clause 18: A process for producing a refined oil product from a biomass, the process comprising: extracting a compound from a biomass charge using a first solvent stream comprising a solvent, the first solvent stream having a first pressure of from 400 psi to 25,000 psi and a first temperature to produce a first oil extract dissolved in the solvent; mixing the first oil extract dissolved in the solvent with a second solvent stream while maintaining the first pressure to form a diluted oil extract; and delivering the diluted oil extract to a filter to separate a byproduct compound from the diluted oil extract to form a dilute refined oil comprising a target compound solubilized in the solvent.

Clause 19: The process of clause 18, wherein the mixing the first oil extract dissolved in the solvent with the second solvent stream comprises maintaining the first pressure while reducing the temperature of the first oil extract dissolved in the solvent below the first temperature by the second solvent stream.

Clause 20: The process of clause 18 or 19, further comprising delivering the dilute oil extract to a heat exchanger to reduce the temperature of the dilute oil extract below the first temperature while maintaining the first pressure to desolubilize the byproduct compound.

Clause 21: A system for extracting a target compound from a biomass, the system comprising: an extraction vessel comprising a biomass charge and supercritical or subcritical CO₂ as a solvent, at a first pressure and a first temperature, to yield a compound-laden solvent stream, the compound-laden solvent stream comprising the solvent, a solubilized target compound, and a solubilized byproduct compound; a heat exchanger to change the temperature while maintaining the first pressure of the compound-laden solvent stream to desolubilize the byproduct compound from the compound-laden solvent stream to yield a resultant stream containing the solvent, the solubilized target compound, and a desolubilized byproduct compound; and a filter medium through which to pass the resultant stream to separate the desolubilized byproduct compound from the solubilized target compound.

Clause 22: A system for extracting a target compound from a biomass, the system comprising: an extraction vessel comprising a biomass charge and supercritical or subcritical CO₂ as a solvent, at a first pressure and a first temperature, to yield a compound-laden solvent stream, the compound-laden solvent stream comprising the solvent, a solubilized target compound, and a solubilized byproduct compound; a mixing point to mix a second solvent stream with the compound-laden solvent stream to change the temperature while maintaining the first pressure of the compound-laden solvent stream to desolubilize the byproduct compound from the compound-laden solvent stream to yield a resultant stream containing the solvent, the solubilized target compound, and a desolubilized byproduct compound; and a filter medium through which to pass the resultant stream to separate the desolubilized byproduct compound from the solubilized target compound.

### BRIEF DESCRIPTION OF THE DRAWINGS

The disclosure will be described with reference to the following drawing figures wherein like reference numbers identify like parts throughout.

FIG. 1 depicts a non-limiting embodiment of the extraction process and system described herein; and

FIG. 2-3 depict photographs of byproduct compounds (waxes) which have been removed following the in-line winterization process described herein.

### DETAILED DESCRIPTION

For the purposes of the following detailed description, it is to be understood that the invention may assume various alternative variations and step sequences, except where expressly specified to the contrary. Moreover, other than in any operating examples, or where otherwise indicated, all numbers expressing, for example, quantities of ingredients used in the specification and claims are to be understood as being modified in all instances by the term “about”. Accordingly, unless indicated to the contrary, the numerical parameters set forth in the following specification and attached claims are approximations that may vary depending upon the desired properties to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard variation found in their respective testing measurements.

Also, it should be understood that any numerical range recited herein is intended to include all sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value of equal to or less than 10.

In this application, the use of the singular includes the plural and plural encompasses singular, unless specifically stated otherwise. In addition, in this application, the use of “or” means “and/or” unless specifically stated otherwise, even though “and/or” may be explicitly used in certain instances. Further, in this application, the use of “a” or “an” means “at least one” unless specifically stated otherwise. For example, “a” wax, “an” extract, and the like refer to one or more of any of these items.

As used herein, the transitional term “comprising” (and other comparable terms, e.g., “containing” and “including”) is “open-ended” and open to the inclusion of unspecified matter. Although described in terms of “comprising”, the terms “consisting essentially of” and “consisting of” are also within the scope of the invention.

As used herein, the term “about” means plus or minus 10% of the numerical value of the number with which it is being used. Therefore, “about 50” means in the range of 45-55.

As used herein, “plant wax” includes one or more of waxes, lipids, wax-like compounds, n-alkanes, paraffins, alkyl esters, or fatty acid compounds that are found in organic plant or fungal material.

### The Compounds

The system of the disclosure can be used to extract any target (desired) compounds from a “charge.” As used herein, a “charge” means a quantity, sample, or other mass of one or more of organic, plant, or fungus materials. The charge can be or include any desirable plant or fungal species, and these are used to extract various useful target compounds. Target compounds include those from at least one of the following plants or trees Cassia, Cinnamon, Sassafras, Camphor, Cedar, Rosewood, Sandalwood, Agarwood, Galangal, Ginger, Basil, Bay Leaf, Buchu, Cannabis, Cinnamon, Sage, Eucalyptus, Guava, Lemon grass, Midaleuca, Oregano, Patchouli, Peppermint, Pine, Rosemary, Spearmint, Tea tree, Thyme, Tsuga, Wintergreen, Benzoin, Copaiba, Frankincense, Myrrh, Chamomile, Clary sage, Clove, Scented geranium, Hops, Hyssop, Jasmine, Lavender, Manuka, Marjoram, Orange, Rose, Ylang-ylang, Bergamot, Grapefruit, Lemon, Lime, Orange, Mango, Tangerine, Valerian, Berries including Allspice and Juniper; Seeds including Anise, Buchu, Celery, Cumin, Nutmeg oil; truffles; or the like. In addition or in the alternative to the specific kinds of plants and trees described above, the charge may also be formed from the wood, rhizomes, resins, peels, flowers, roots, stems, bark, leaves, or any other parts of organic materials, plant materials, or fungus materials, or combinations of any of the above.

In some embodiments, the charge may be formed from plant materials. Examples of plant materials that may be used for the charge are not limited, and include cannabis sativa, cannabis indica, and cannabis ruderalis (collectively referred to as “cannabis” throughout the disclosure), including varieties that are cultivated for medical, industrial, textile, fuel, paper, chemical, food, and recreational purposes, among other uses. When the charge is cannabis, it may include any part of the cannabis plant, including the stems, leaves, seeds, flowers, buds, roots, or combinations thereof. In some embodiments, the plant charge of cannabis is used for the extraction of various useful target compounds, including cannabinoids, tetrahydrocannabinol (THC), cannabinoid isomers, cannabinoid stereoisomers, tetrahydrocannabinolic acid (THCA), cannabidiol (CBD), cannabidiolic acid (CBDA), cannabinol, (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannebielsoin (CBE), cannabicitran (CBT), and combinations or derivatives of the above. In addition to these cannabinoid compounds the plant charge of cannabis can also be used for the extraction of light volatile compounds such as monoterpenes, sesquiterpenes, tri-terpenes, and other terpenoid compounds.

While the above plants and compounds are described, the extraction systems and processes of the disclosure and its use of CO₂ solvent is not limited in application and can be used to extract any useful compound from any plant or fungal charge that is placed within it. In particular, volatiles, oils, resins, acids, bases, aqueous solutions, and other compounds are all contemplated for extraction by the disclosed extraction systems and processes. Therefore, the extraction system and process described herein may also be used to extract target compounds from pre-extracted oils as well.

### Extraction Process and System

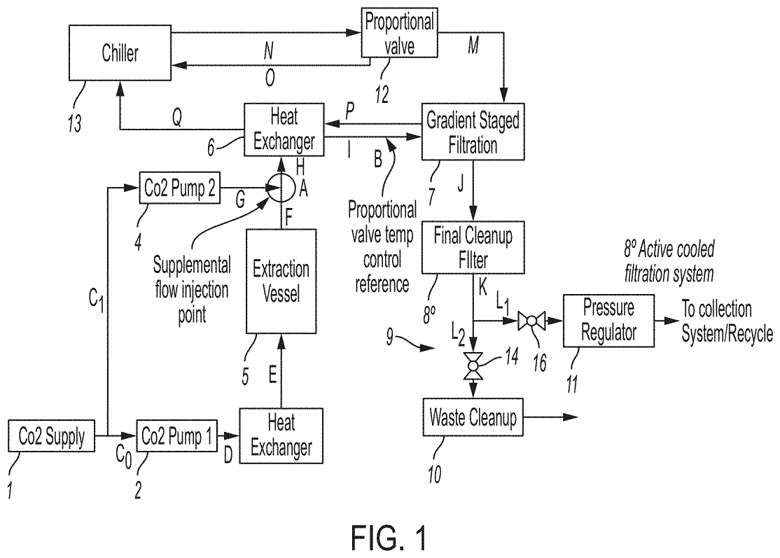

Referring now to FIG. 1, an extraction flow diagram is disclosed. As shown, there is an extraction, a winterization, and a filtration phase.

In the extraction phase, a primary CO₂ pump (2) may flow CO₂ solvent into and through an extraction vessel (5) containing a biomass charge (not shown) in which the CO₂ solvent is maintained at a supercritical or subcritical liquid state utilizing a pressure regulator (11) at the outlet of the winterization system. The supercritical or subcritical CO₂ solvent may contact the biomass charge and thereby become laden with charge compounds, which includes various target (desirable) compounds but which also contains byproduct (undesirable) compounds such as, but not limited to, plant wax, lipids, and wax-like compounds to be removed in a filtering process. Therefore, the compound-laden solvent stream (F) comprises the solvent, the solubilized target compound, and the solubilized byproduct compound. The compound-laden solvent stream (F) may also be referred to interchangeably as an oil extract dissolved in the solvent.

The CO₂ solvent may be used to extract compounds from the biomass charge in the extraction vessel (5) at a first temperature and a first pressure.

The first temperature may range from −60° C. to 200° C., such as from −60° C. to 100° C., from 0° C. to 100° C., from 32° C. to 100° C. (e.g., with a supercritical CO₂ solvent at 1,080 psi), or from 20° C. to 32° C. (e.g., with a subcritical CO₂ solvent at 1,080 psi).

The first pressure may range from 400 psi to 25,000 psi, such as from 400 to 20,000 psi, from 400 to 10,000 psi, from 800 to 7,500 psi, from 800 to 6,000 psi, from 800 to 5,000 psi, or from 1,000 to 10,000 psi. The first pressure may be maintained throughout the process until the target compound is separated from the CO₂ solvent. The first pressure may be maintained in the extraction vessel (5), at the supplemental flow injection point (A) of a second solvent stream (G) with the compound-laden solvent stream (F), and at the heat exchanger (6) to prevent or reduce undesired, pre-mature precipitation of the target compound. By the pressure being “maintained”, it is understood to mean having the same target pressure (targeted by the pressure regulator (11) through the various stages), and it will be understood that minor drops in pressure may occur as a result of flow of the stream through the system (e.g., the tubes thereof) without the pressure not being “maintained”. These minor pressure drops caused by unavoidable friction occurring in the system may amount to less than 20%, such as less than 10%, relative to the pressure in the extraction vessel (5) of the system.

The first temperature may be from 0° C. to 100° C., and the first pressure may be from 800 psi to 7,500 psi.

After the extraction phase, in the winterization phase, the first temperature may be changed for the compound-laden solvent stream (F) while maintaining the first pressure. The change (e.g., decrease) in temperature while maintaining the first pressure may desolubilize the byproduct compound from the compound-laden solvent stream (F), while preventing or reducing undesired, pre-mature precipitation of the target compound. The decrease in temperature while maintaining the pressure may yield a resultant stream containing the solvent, the solubilized target compound, and a desolubilized byproduct compound.

As part of this winterization phase, a second solvent stream (G) may optionally be injected and combined with the compound-laden solvent stream at the supplemental flow injection point (A) (also referred to as a mixing point) to dilute the compound-laden solvent stream (F). The second solvent may comprise supercritical or subcritical liquid CO₂. The second solvent may have a temperature below the temperature of the compound-laden solvent stream (F). The second solvent may have a temperature identical to the temperature of the compound-laden solvent stream (F). The first pressure may be maintained at this step. The combination of the second solvent stream (G) with the compound-laden solvent stream (F) to form the diluted compound-laden solvent stream (H) (also referred to as the diluted oil extract) may decrease the temperature of the compound-laden solvent stream (F) to desolubilize the byproduct compound from the diluted compound-laden solvent stream (H).

The first pressure may be maintained at this step of injecting the second solvent stream to prevent any drop off (precipitation) in target compounds. If the second solvent stream is introduced after the pressure has been dropped, a significant number of compounds (including target compounds) may drop off for two reasons: (1) the CO₂ density drops causing the CO₂ solvent to solute ratio to drop; and (2) there is a natural cooling effect that happens which reduces solubility for a lot of compounds. Once compounds have dropped out of a solution, it can be harder to dissolve them again. Adding the second solvent stream with the first pressure maintained as described herein (when the compounds are still dissolved) serves to dilute the stream further, then dewaxing the oil while all of the compounds are still in solution.

As a non-limiting theoretical example according to the process of the present disclosure in which the first pressure is maintained, if an extraction is performed at 3,000 psi at 70° C., the CO₂ density is 0.67 g/cc. If the solution is 1% extracted compounds by mass, then there are 0.0067 g of extracted compounds per ml. The solvent to solute ratio is 100:1. When the second solvent stream is injected into the solution, the CO₂ would be at the same pressure, but at 20° C.—the density of the second solvent stream CO₂ is 0.94 g/cc. When this second solvent stream is added, the solvent to solute ratio is now 240:1 before the temperature is dropped to dewax the oil. The compounds are able to stay in solution according to this process.

Conversely, as a non-limiting theoretical example according to a process in which the first pressure is not maintained, using the same extraction conditions, the CO₂ density is again 0.67 g/cc. However, dropping the pressure and the temperature prior to dewaxing causes a typical density at the first point of depressurization to be in the range of 0.3 g/cc. In such case, the solvent ratio is 45:1 before the second solvent is added, at which conditions a lot of compounds will already drop out of solution.

The amount of the second solvent stream (G) is not limited and can be changed depending on the pressure, temperature, and the compounds being extracted. The second solvent stream (G) may be substantially not laden with (containing only trace amounts) or be free of compounds and when it is combined with the compound-laden solvent stream (F), the combined diluted compound-laden solvent stream (H) may have an increased solubility for the target compounds. This injection may happen after the compound-laden solvent stream (F) leaves the extraction vessel (5) and the pressure conditions in the supplemental flow injection point (A) may be the same as those set in the extraction vessel (5). The mixing of these two solvent streams may happen prior to any active cooling of the compound-laden solvent stream (F) leaving the extraction vessel (5) to maximize the compound solubility and to prevent rapid desolubilization due to retrograde solubility effects once the active cooling stages are introduced. The second solvent stream (G) may be provided via a secondary CO₂ pump (4), or a single pump with a proportioning valve injects the second solvent, such as supercritical or subcritical CO₂ solvent, at the extraction vessel's (5) pressure to maintain proper compound solubility, while also maximizing the heat transfer efficiency, before being introduced to the active cooling process (e.g., the heat exchanger (6)) and/or eventual filtration system (7-8).

As part of this winterization phase, the compound-laden solvent stream (F) or optionally the diluted compound-laden solvent stream (H) may proceed through a cooling heat exchanger (6) while still maintaining the first pressure set in the extraction vessel (5). This heat exchanger (6) may be the first step in the active cooling process, and it may take place at the same first pressure as the extraction vessel (5). The use of the heat exchanger (6) may decrease the temperature of the compound-laden solvent stream (F) (or its diluted stream (H)) to desolubilize (or further desolubilize) the byproduct compound therefrom.

In the heat exchanger (6), the temperature of the compound-laden solvent stream (F) (or its diluted stream (H)) may be reduced to −10° C. to 15° C., such as from 0° C. to 6° C., while maintaining the pressure at the first pressure.

In this winterization phase, the step of combining the compound-laden solvent stream (F) with the second solvent stream (G) may be included without inclusion of the heat exchanger (6) step. Alternatively, in this winterization phase, the step of using the heat exchanger (6) may be included without combining the compound-laden solvent stream (F) with the second solvent stream step. Alternatively, in this winterization phase, both the combining the compound-laden solvent stream (F) with the second solvent stream (G) step and the using the heat exchanger (6) step may be included in sequence.

From the winterization phase, a resultant stream may be formed containing the solvent, the solubilized target compound, and the desolubilized byproduct compound.

After the winterization phase, in the filtration phase, the filtration system (7-8) may be configured to separate the resultant stream, such as separating the desolubilized byproduct compound from the solubilized target compound. This process may be conducted at the same first pressure as the extraction vessel (5) and winterization process, and the filtration system (7-8) may encompass two separate filtration vessels. A primary filter vessel (7) may include a Gradient Stage Filtration Unit (GSFU) that has a gradient filter stack system that removes the plant wax wastes (a byproduct compound). The relatively large pore size of the primary filter vessel (7) may capture the majority of the wax without clogging of the apparatus. The second filter vessel (8) may be a Final Clean-up Filter Unit (FCFU) that is smaller in size and volume (compared to the primary filter vessel (7)) and may house an ultra-fine micron filter as a final step to remove any remaining plant wax waste (a byproduct compound) that has made it through the GSFU unit. It will be appreciated that additional filtration units may be employed for desired results, or a single filtration unit may be employed. Both vessels (7-8) may be actively cooled and feature little dead volume, a feature for both maintaining solubility of the desired compounds as well as for the overall thermodynamic load of the system. The stream that outputs from the filtration system (7-8) may contain solubilized target compounds and be substantially free (containing only trace amounts) or free of byproduct compounds, such as plant waxes and wax-like compounds. This stream of solubilized target compounds (also referred to as a dilute refined oil or refined oil) may be collected and sent to the collection system/recycle portion. Thus, the filtration system (7-8) may separate and filter the byproduct compounds from the target compounds.

The solubilized target compound leaving the filtration system (7-8) may be processed to isolate the target compounds from the solvent. Isolating the target compounds from the solvent may comprise reducing the pressure (and/or the temperature) to cause the solubilized target compound (such as CBD, THC, other cannabinoids, terpenes, terpenoid compounds or other botanical compounds) to be released separately into the target compound and the solvent. The pressure may be reduced by flowing the stream through a pressure regulator (11). Thus, the pressure reduction to isolate the target compound from the solubilized target compound may occur after the solubilized target compound leaves the filtration system (7-8). This isolated target compound may be substantially free (containing only trace amounts) or free of byproduct compounds (e.g., waxes or lipids).

While these connections are provided by way of example, the connections to the in-line winterization system are not limited and other connections to other devices can also be made. One or more additional devices including valves, heaters, sensors, and other devices can be included by connecting them to the in-line winterization system, and in some embodiments such devices can be positioned in the lines that connect the in-line winterization system to other parts of the overall extraction system.

It should be noted that in certain other embodiments, the filtration system only has a single filter. In such designs, the surface area of the single filter may be sufficient to remove the byproduct compounds.

By way of the additional unsaturated CO₂ (the second solvent stream (6)) from the secondary CO₂ pump (4) and/or the heat exchangers (6) contained within the in-line winterization system, the temperature and pressure within both vessels that comprise the filter units (7, 8) may be maintained at about −60° C. to about 100° C. and between about 400 pounds per square inch to about 25,000 pounds per square inch (about 2,758 kPa to about 172,369 kPa). In some embodiments, the temperature is about −10° C. to about 15° C. and the pressure is about 1100 psi to about 10,000 psi. The pressure may be homogenous (maintained) throughout the in-line winterization system, and the selected process and apparatus ensures that that target compounds such as CBD, THC, other cannabinoids, terpenes, terpenoid compounds, and other botanical compounds remain soluble within the CO₂, while byproduct compounds such as the plant wax waste is filtered out from the compound-laden solvent stream (F).

The design of the in-line winterization system is not limited but is configured to achieve the removal of the various plant waxes and wax like byproduct compounds. The system may be configured to be easily cleaned in place by means of a supercritical CO₂ cleaning method which may remove the byproduct compounds from the filtration system (7-8) and then precipitate them into a waste/cleanup vessel (10) that is removed from the process solvent stream to avoid cross contamination. In addition to the system's cleaning method the filter elements of both the GSFU (7) and FCFU (8) vessels can be accessed by the end user for removal and servicing.

### EXAMPLES

Various experimental Examples were performed to test different setpoints and design iterations of the in-line winterization vessel. In the Examples, acetone tests were performed to qualitatively test for n-alkane wax contamination in the final product. In the Examples, the test procedures included one or more temperature offsets applied to the chillers (and thus the coolant in the heat exchanger) so that the in-line winterization system had the correct target inlet and outlet temperatures.

Experimental information for the multistep filtration vessels are depicted below in Table 1.

TABLE 1
Filtration Unit

| Parameter | Value |
|---|---|
| Filter Vessels' Inlet Temperature: | −60° C. to 200° C. |
| Filter Vessels' Outlet Temperature: | −60° C. to 200° C. |
| Filter Vessels' Pressure | 400-25,000 psi |
| GSFU Filter Size | 5000 μm to 1.00 nm |
| FCFU Filter Size | 5000 μm to 1.00 nm |
| CO₂ Phase | Liquid or Gas |

### Exemplary Extraction Process:

Clean system thoroughly including both a cleaning run and by wiping down the vessels with alcohol or d-limonene.

Start in-line winterization system chiller(s) and set the temperature for the winterization process.

Weigh out total cannabis, hemp, or other botanical biomass to be extracted (the biomass charge).

Load the biomass charge about 300 g at a time stopping between each loading step to pack the biomass charge down with the steel rod.

Seal the extraction vessel and check all valves.

Start extraction run.

Collect extract upon the completion of the extraction run.

After collection of the botanical compounds, set the system to conduct a cleaning flush of the waxes from the lines and multistep filtration vessels bypassing the extraction vessel in the process.

Depressurize system and remove the raffinate from the extraction vessel.

Collect the waxes from the wax recovery vessel and prepare the system for the next run.

Clean in-line winterization system, housings, lines and filters as needed.

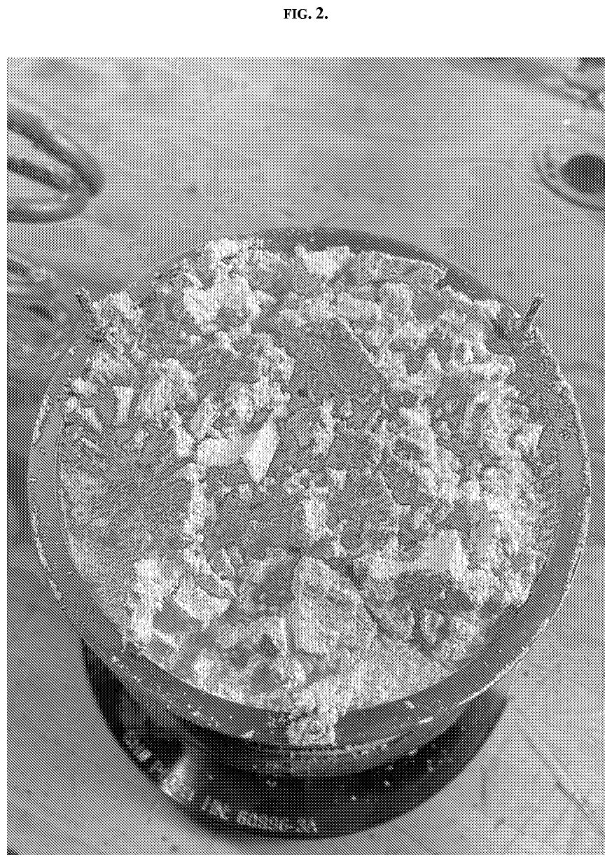

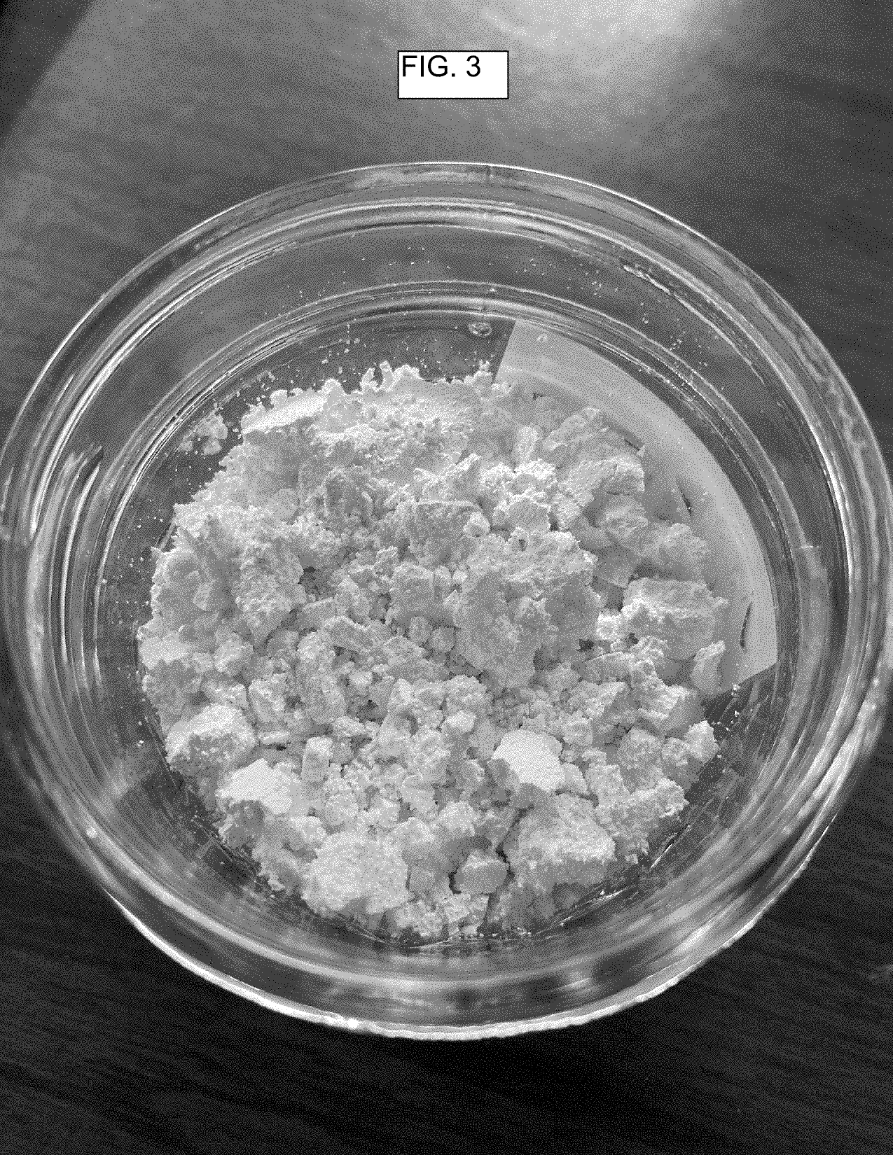

FIGS. 2-3 depict photographs of byproduct compounds (waxes) which have been removed following the in-line winterization described herein. FIG. 2 demonstrates the filter as being completely covered in the waxes and devoid of the desired botanical compounds.

In the foregoing process of the Example, the steps are performed in the listed order. However, the disclosure is not so limited, and a different order can be performed. Following the process of the Example, a waxless oleoresin extract was collected in any of the three collection vessels, and at least one plant wax was collected from the in-line winterization vessel.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only and is not intended to be limiting.

What is claimed is:

1. A process for extracting a target compound from a biomass, the process comprising:
extracting a compound from a biomass charge using supercritical or subcritical CO₂ as a solvent, at a first pressure and a first temperature, to yield a compound-laden solvent stream, the compound-laden solvent stream comprising the solvent, a solubilized target compound, and a solubilized byproduct compound;
changing the temperature while maintaining the first pressure of the compound-laden solvent stream to desolubilize the byproduct compound from the compound-laden solvent stream to yield a resultant stream containing the solvent, the solubilized target compound, and a desolubilized byproduct compound;
passing the resultant stream through a filter medium to separate the desolubilized byproduct compound from the solubilized target compound; and
collecting the solubilized target compound.

2. The process of claim 1, further comprising diluting the compound-laden solvent stream with a second solvent stream while maintaining the first pressure.

3. The process of claim 2, further comprising decreasing the temperature of the diluted compound-laden solvent stream while maintaining the first pressure to desolubilize the byproduct compound from the diluted compound-laden solvent stream.

4. The process of claim 2, wherein the second solvent stream comprises supercritical or subcritical CO₂.

5. The process of claim 1, wherein the biomass charge is selected from a plant or fungal species.

6. The process of claim 1, wherein the biomass charge comprises cannabis.

7. The process of claim 1, wherein the target compound is extracted from at least one of plants or trees including Cassia, Cinnamon, Sassafras, Camphor, Cedar, Rosewood, Sandalwood, Agarwood, Galangal, Ginger, Basil, Bay Leaf, Buchu, Cannabis, Cinnamon, Sage, Eucalyptus, Guava, Lemon grass, Midaleuca, Oregano, Patchouli, Peppermint, Pine, Rosemary, Spearmint, Tea tree, Thyme, Tsuga, Wintergreen, Benzoin, Copaiba, Frankincense, Myrrh, Chamomile, Clary sage, Clove, Scented geranium, Hops, Hyssop, Jasmine, Lavender, Manuka, Marjoram, Orange, Rose, Ylang-ylang, Bergamot, Grapefruit, Lemon, Lime, Orange, Mango, Tangerine, Valerian, Berries including Allspice and Juniper; Seeds including Anise, Buchu, Celery, Cumin, Nutmeg oil; or truffles.

8. The process of claim 1, wherein the target compound is selected from at least one of cannabinoids, tetrahydrocannabinol (THC), cannabinoid isomers, cannabinoid stereoisomers, tetrahydrocannabinolic acid (THCA), cannabidiol (CBD), cannabidiolic acid (CBDA), cannabinol, (CBN), cannabigerol (CBG), cannabichromene (CBC), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannebielsoin (CBE), cannabicitran (CBT), or combinations or derivatives thereof.

9. The process of claim 1, wherein the byproduct compound comprises a wax or a lipid.

10. The process of claim 1, wherein the first temperature is from −60° C. to 200° C.

11. The process of claim 1, wherein the first pressure is from 400 psi to 25,000 psi.

12. The process of claim 1, wherein the first temperature is from 0° C. to 100° C., and the first pressure is from 800 psi to 7,500 psi.

13. The process of claim 1, wherein the changing the temperature comprises decreasing the temperature to from −10° C. to 15° C.

14. The process of claim 1, wherein the changing the temperature comprises decreasing the temperature to from 0° C. to 6° C.

15. The process of claim 1, further comprising reducing the pressure of the solubilized target compound to release the target compound from the solvent.

16. The process of claim 15, wherein the released target compound is free of the byproduct compound.

17. A target compound extracted from a biomass charge according to the process of claim 1, wherein the target compound is free of a wax or a lipid.

18. A process for producing a refined oil product from a biomass, the process comprising:
extracting a compound from a biomass charge using a first solvent stream comprising a solvent, the first solvent stream having a first pressure of from 400 psi to 25,000 psi and a first temperature to produce a first oil extract dissolved in the solvent;
mixing the first oil extract dissolved in the solvent with a second solvent stream while maintaining the first pressure to form a diluted oil extract; and
delivering the diluted oil extract to a filter to separate a byproduct compound from the diluted oil extract to form a dilute refined oil comprising a target compound solubilized in the solvent.

19. The process of claim 18, wherein the mixing the first oil extract dissolved in the solvent with the second solvent stream comprises maintaining the first pressure while reducing the temperature of the first oil extract dissolved in the solvent below the first temperature by the second solvent stream.

20. The process of claim 18, further comprising delivering the dilute oil extract to a heat exchanger to reduce the temperature of the dilute oil extract below the first temperature while maintaining the first pressure to desolubilize the byproduct compound.
